(19) United States
(12) Patent Application Publication
(10) Pub. No.: US 2024/0196983 A1
(43) Pub. Date: Jun. 20, 2024

(54) Liquid Jet Inhalation Device

(71) Applicant: JT International SA, Geneva (CH)

(72) Inventor: Alec Wright, Guildford (GB)

(73) Assignee: JT International SA, Geneva (CH)

(21) Appl. No.: 18/286,183

(22) PCT Filed: Apr. 29, 2022
(86) PCT No.: PCT/EP2022/061513

§ 371 Date: Oct. 9, 2023

(30) Foreign Application Priority Data
Apr. 30, 2021 (EP) 21171519.8

(51) Int. Cl. A24F 40/51; A24F 40/10; A24F 40/48

(52) U.S. Cl. CPC A24F 40/51; A24F 40/10; A24F 40/48

(57) ABSTRACT

An inhalation device for generating from a liquid a vapour that can be inhaled by a user includes a compartment for holding a reservoir arranged to store an amount of a liquid to be vapourized, at least one liquid jet device for producing on demand drops of the liquid, a liquid conduit for guiding an amount of the liquid to the liquid jet device and arranged on one side of the compartment, a sensor arranged to detect a situation indicative of whether the liquid conduit can supply liquid to the liquid jet device and arranged on an opposite side of the compartment, and a control unit configured to control the liquid jet device based on the output of the sensor.

### TECHNICAL FIELD

The present invention relates to inhalation devices with at least one liquid jet device for producing drops of a liquid on demand. More particularly, the present invention relates to such inhalation devices in the form of electronic cigarettes, cigalikes, e-cigarettes, vapour inhalers and related devices.

### BACKGROUND

In the arts there are several types and concepts for range of purposes broad inhalation devices that serve a including medical and therapeutic applications and also leisure and pleasure devices such as electronic cigarettes. Existing inhalation devices either change the phase of a fluid before inhalation with for example a wick and a coil so as to significantly raise the vapor temperature above human body temperature or deliver drops a room temperature by, for example, employing an ultrasonic mesh. In the above wick and coil system the vapor can be perceived as ‘warm’ by a user in the mouth, whereas in the ultrasonic mesh systems, the vapor is usually perceived as ‘cold’.

Such inhalation devices are oftentimes portable and pocket-size devices that can easily fit in the user's hand or can be handled by the user's fingers alone. In this way, the devices can be convenient for use and can be carried by a user for regular or emergency use. In the former case, a user can conveniently carry an inhalation device in the form of an e-cigarette to use and enjoy it whenever and wherever desired, whereas in the latter case a medical or therapeutic inhalation device may be ready to use when needed.

As such devices generate from a liquid drops or vapour that is to be inhaled by a user, the reliability and the fidelity with regard to droplet and vapour generation can be important. Namely, in a case in which only limited liquid supply is available a droplet may be formed from a target ejection power but only consisting of a reduced volume due to an obstruction or low running of a liquid supply. For example, then the temperature of the droplet may be too high or at least out of control. Such disadvantages may lead to further undesirable effects when using an inhalation device.

There is therefore a need for improved inhalation devices that not only ensure convenience in use and carriage, but also improve quality of experience and fidelity of action. It is thus an object of the present invention to provide such improved inhalation devices that can remedy the drawbacks of the conventional solutions.

### SUMMARY

The mentioned drawbacks are remedies by the subject-matter of the independent claims. Further preferred embodiments of the present invention are defined in the dependent claims.

According to one embodiment of the present invention there is provided an inhalation device for generating from a liquid a vapour that can be inhaled by a user, said inhalation device comprising a compartment for holding a reservoir arranged to store an amount of a liquid to be vapourized; at least one liquid jet device for producing on demand drops of said liquid; a liquid conduit for guiding an amount of said liquid to said liquid jet device and arranged on one side of said compartment; a sensor arranged to detect a situation indicative of whether the liquid conduit can supply liquid to the liquid jet device and arranged on an opposite side of said compartment; and a control unit configured to control said liquid jet device based on the output of said sensor.

### BRIEF DESCRIPTION OF THE DRAWINGS

Embodiments of the present invention, which are presented for better understanding the inventive concepts and which are not to be seen as limiting the invention, will now be described with reference to the Figures in which:

FIGS. 1A to 1C show schematic views of an inhalation device according to respective embodiments of the present invention;

FIG. 2 shows a schematic side view of an inhalation device according to an embodiment of the present invention;

FIG. 3 shows a schematic view of possible orientations of an inhalation device relative to a horizontal base line;

FIG. 4A shows a schematic view of a MEMS layout in a substrate according to a respective embodiment of the present invention;
and

FIG. 4B shows a schematic view of a vapour generator assembly of an inhalation device according to respective embodiments of the present invention.

### DETAILED DESCRIPTION

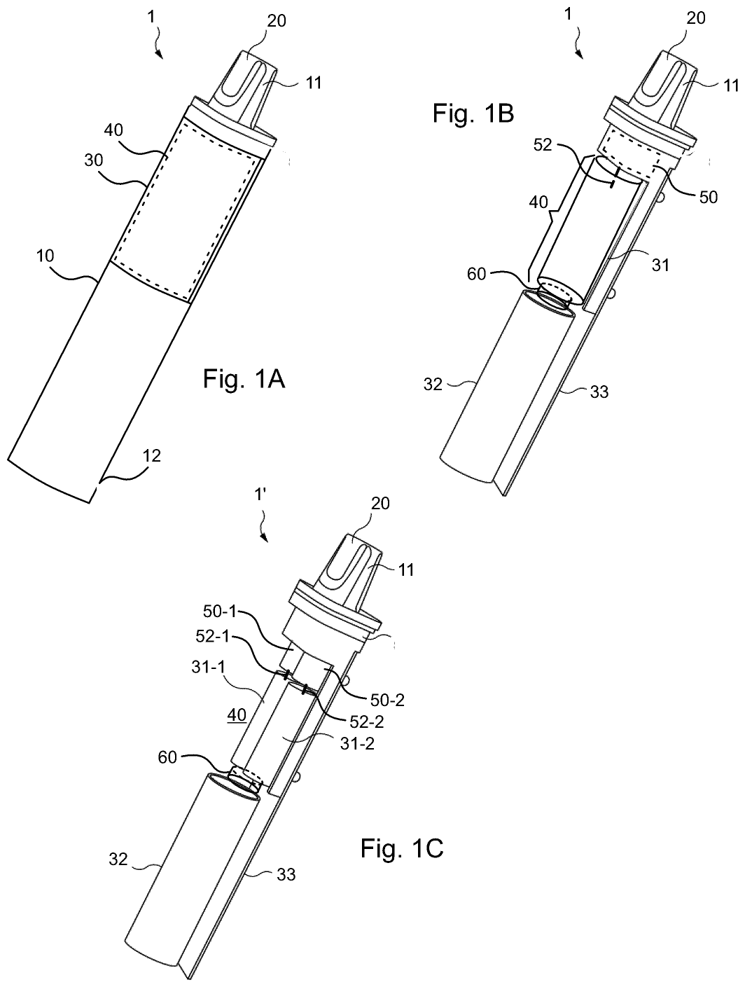

FIGS. 1A to 1C show schematic views of an inhalation device according to respective embodiments of the present invention. FIG. 1A shows the inhalation device 1 in a closed and ready to use state with an elongate housing 10 which provides a proximal end 11 and a distal end 12. Toward the proximal end 11 there is arranged a mouthpiece 20 through which a user can inhale a generated vapour. A cover 30 may render accessible a compartment that holds a reservoir arranged to store an amount of a liquid to be vapourized. Further details can be explained better when the internals of the inhalation device are shown.

FIG. 1B shows a schematic view of the inhalation device 1 according to the respective embodiment of the present invention in an state in which the internal opened configuration can be rendered visible. The inhalation device 1 is generally for generating from a liquid a vapour that can be inhaled by a user, for example by means of the mouthpiece 20 arranged toward the proximal end 11 of the elongate housing 10. The inhalation device 1 comprises a compartment 40 for holding or accommodating a reservoir 31 arranged to store an amount of a liquid to be vapourized. The reservoir 31 can be fixed to the inhalation device 1 and be either in a single-fill configuration or in a refillable configuration. In related embodiments, the reservoir 31 is part of a cartridge that can be replaceably inserted into the compartment 40 in the form of a consumable.

The inhalation device 1 further comprises at least one liquid jet device 50 for producing on demand drops of said liquid. A a liquid conduit 52 is arranged for guiding an amount of said liquid to said liquid jet device 50 and arranged on one side of said compartment 40. In the shown embodiment, this one side is toward the proximal end 11 of the elongate housing 10. The inhalation device comprises further a sensor 60 that is arranged to detect a situation indicative of whether the liquid conduit 52 can supply liquid to the liquid jet device 50 and arranged on an opposite side of said compartment 40. Specifically, in the shown embodiment, this opposite side is toward the distal end 12 of the elongate housing of the inhalation device 1. Further, there is provided a control unit which is configured to control said liquid jet device 50 based on the output of said sensor 60. In the shown embodiment, a corresponding control unit may be implemented as one or more microprocessor (s) mounted on a printed circuit board (PCB) 33. A power source 32 is also provided for supplying electrical energy to the liquid jet device(s), the control unit, the sensor, and any other electric or electromechanical component.

FIG. 1C shows a schematic view of the inhalation device 1′ according to a further embodiment of the present invention in an opened state in which the internal configuration can be rendered is visible. It noted that from the external appearance the device l′ is identical to, very similar to or at undistinguishable from the device 1 shown in FIG. 1A. In this embodiment, the inhalation device l′ comprises more than one liquid jet device for vapour generation, namely the liquid jet devices 50-1 and 50-2 that are supplied by one or several liquids from reservoirs 31-1 and 31-2 through respective liquid conduits 52-1 and 52-2. However, also this configuration considers the arrangement of the liquid jet devices 50-1, 50-2 at one side of the compartment 40. In the shown embodiment, this one side is toward the proximal end 11 of the elongate housing 10. Also, there is considered the arrangement of a sensor 60 on the opposite side of the compartment 40, which is, in the present embodiment, toward the distal end 12 of the elongate housing of the inhalation device 1.

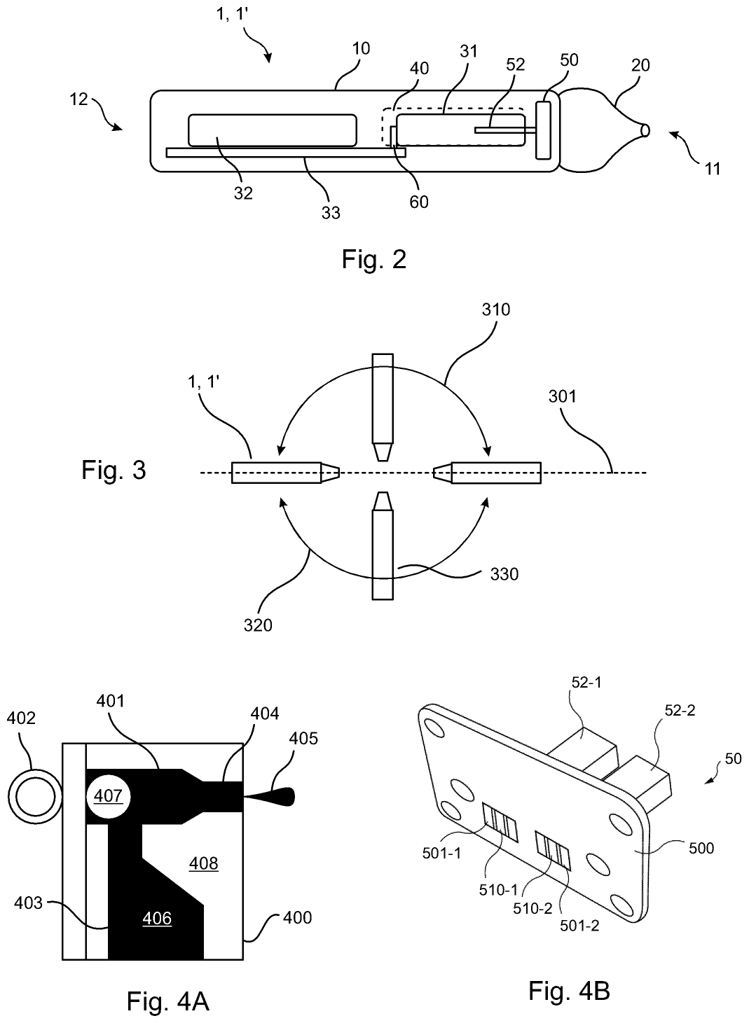

FIG. 2 shows a schematic side view of an inhalation device according to an embodiment of the present invention. In this view the inhalation device 1 comprises an elongate housing 10 with a proximal end 11 and a distal end 12, wherein the at least one liquid jet device 50 and the liquid conduit 52 are arranged on a side of said compartment 40 toward the proximal end 11. The liquid jet device 50 may comprise or may be associated with a filter, and the liquid conduit 52 may comprise or may be in the form of a needle that may be arranged to penetrate a shell of a reservoir 31 in the form of a capsule or consumable. A sensor 60 is arranged on the opposite side of said compartment 40 toward the distal end 12 of the elongate housing 10.

Generally, the sensor 60 may comprise any one of an orientation sensor arranged to detect an orientation of the inhalation device 10, an accelerometer, a gyroscope, a gravity sensor, a liquid sensor arranged at a bottom end of said reservoir 31, a capacitance sensor, a light sensor, and an ultrasonic sensor. Generally, the control unit can be configured to deactivate operation of said liquid jet device 50 if the sensor 60 detects a situation indicative of that the liquid conduit cannot supply liquid to the supply channel. Specifically, the sensor 60 may detect a situation indicative of that the orientation of the inhalation device 1 is approximately horizontal or approaching that horizontal orientation, wherein such an orientation can be identified as a boundary between “admissible” orientations in which reliable operation of the device can be assumed, and “non-admissible” orientations in which operation of the device should be avoided. Preferably, the non-admissible range may commence at angles of more than 60 degrees, the orientation being measured between a main axis along the longitudinal extension of the inhalation device and a normal to ground. If the device is held at such orientations it may become difficult for the liquid conduit to reliably be provided with liquid, so that any liquid jet device may run dry and disadvantages may occur: For example, a reduced volume of liquid ready to be expelled may be heated up too much so that too hot or too fast droplets may be ejected. This may lead in undesirable effects relating to taste, fidelity and safety. Further, also damage may occur to the liquid jet device, any parts thereof or also other parts of the inhalation device that may get into contact with the droplets.

The latter is shown and explained in FIG. 3 of a schematic view of possible orientations of an inhalation device 1, 1′ relative to a horizontal base line 301. Specifically, the device 1, 1′ may be orienteered relative to the horizontal base line 301 in a range 320 in which operation can be recommended. This recommended range may include an ideal or optimum orientation 330 which is approximately vertical. Further, there may be a range 310, in which operation of the liquid jet device, and, with this, the inhalation device may be unreliable and thus operation should not be permitted.

A capacitive sensor in the liquid reservoir can detect the presence of the liquid. By placing the sensor near the bottom of the reservoir when full of the liquid in any orientation it can detect the presence of the liquid. When the liquid level is low and the device is the upward-facing orientation it will still detect the liquid. However, when the liquid is low and the device is inverted it will not detect liquid. It is in this scenario that the device will struggle to pull the liquid from the reservoir and a dry puff is possible. Therefore, if the sensor does not detect any liquid it prevents the device from firing (Device firing is when the aerosol is being produced by the die/MEMS), and thus damaging the MEMS. The reason for the disallowed puff could be informed to the user through haptic feedback or an LED.

In a separate arrangement it is possible to use an accelerometer in the device to determine the orientation of the device. When the device is in any position other than the designated vertical one it will prevent the device from firing. It is also possible to combine both the capacitive sensor and the accelerometer as a second level of verification. The capacitive sensor can check the liquid level and the accelerometer the orientation of the device. The control unit (MCU) can then evaluate the date from both these sensors and decide if the device should fire or not.

Generally, the inhalation device relies on a liquid jet device comprising a firing chamber, an ejection nozzle and a supply channel embedded in a substrate, the supply channel being in fluid communication with the liquid conduit. A specific embodiment shows FIG. 4A with a schematic view of a MEMS layout in a substrate according to a respective embodiment of the present invention. The liquid jet device 400 is formed as a MEMS in a substrate 408 of any suitable material, for example silicon. In that substrate 408 there is formed a fluid chamber 401, an ejection nozzle 404, and a supply channel 403 in liquid communication with a reservoir for providing liquid 406 to the fluid chamber 401 so as to be vaporized or atomized. An ejection heating element 402 is arranged in the vicinity of the fluid chamber 401 so as to heat up a portion of the liquid 46 to vaporized and form a gas bubble 407. Specifically, said ejection heating element 402 can be a resistor arranged in a vicinity of said fluid chamber 401 and configured to heat a first amount of the liquid 406 to at least a vaporization temperature, so that a vapour bubble expels a drop of the liquid through the ejection nozzle. So, the resulting expansion leads to the ejection of an amount of the liquid in the form of a drop or droplet 405 that then can form in the mixing chamber a vapour or aerosol.

FIG. 4B shows a schematic view of a vapour generator assembly of an inhalation device according to respective embodiments of the present invention. This vapour generator assembly 50 employs liquid jet devices for generating drops from a liquid to be mixed with air so as to be inhaled as a vapour by a user. The vapor generation unit 50 comprises here two microfluidic structures or MEMS dies 510-1, 510-2. The two MEMS dies 510-1, 510-2 are fastened, e.g. soldered, to the printed circuit board 500. Each MEMS 510-1, 510-2 has an upper surface 501-1, 501-2. The two upper surfaces 501-1, 501-2 can be coplanar and thus form the upper surface of the vapor generation unit 50. On the opposite side of the printed circuit board 500, the vapor generation unit 50 comprises two inlet ports with a fluid conduit 52-1, 52-2. Each inlet port is configured to be fluidically connected to an inner volume of the reservoir.

In this way, the disclosed embodiments can obtain one or more of the following aspects and advantages. As a MEMS in the device can generate the aerosol for the user to inhale is housed within the device. The MEMS and/or the capsule may not be disposable, which means that maintaining the reliability of the MEMS can be important so that it does not limit the lifespan of the product. Dry firing may be one way of causing potential damage to the device. This situation is more likely if the device is inverted so that the liquid supply from the reservoir to the MEMS is limited. Hardware and firmware designs that reduce this probability are therefore adventurous for the device.

Although detailed embodiments have been described, these only serve to provide a better understanding of the invention defined by the independent claims and are not to be seen as limiting.

1. An inhalation device for generating from a liquid a vapour that can be inhaled by a user, said inhalation device comprising:
a compartment for holding a reservoir arranged to store an amount of a liquid to be vapourized;
at least one liquid jet device for producing on demand drops of said liquid;
a liquid conduit for guiding an amount of said liquid to said at least one liquid jet device and arranged on one side of said compartment;
a sensor arranged to detect a situation indicative of whether the liquid conduit can supply liquid to the at least one liquid jet device and arranged on an opposite side of said compartment; and
a control unit configured to control said at least one liquid jet device based on an output of said sensor.

2. The inhalation device according to claim 1, further comprising an elongate housing with a proximal end and a distal end, wherein the at least one liquid jet device and the liquid conduit are arranged on a side of said compartment toward the proximal end, and wherein the sensor is arranged on the opposite side of said compartment toward the distal end of the elongate housing.

3. The inhalation device according to claim 1, wherein said at least one liquid jet device comprises a firing chamber, an ejection nozzle and a supply channel embedded in a substrate, the supply channel being in fluid communication with the liquid conduit.

4. The inhalation device according to claim 3, wherein the at least one liquid jet device is in the form of a micro-electromechanical system, MEMS.

5. The inhalation device according to claim 1, wherein the sensor comprises an orientation sensor arranged to detect an orientation of said inhalation device.

6. The inhalation device according to claim 5, wherein said orientation sensor comprises any one of an accelerometer, a gyroscope, and a gravity sensor.

7. The inhalation device according to claim 1, wherein the sensor comprises a liquid sensor arranged at a bottom end of said reservoir.

8. The inhalation device according to claim 7, wherein said liquid sensor comprises any one of a capacitance sensor, a light sensor, and an ultrasonic sensor.

9. The inhalation device according to claim 1, wherein said control unit is configured to deactivate operation of said at least one liquid jet device when the sensor detects a situation indicative of that the liquid conduit cannot supply liquid to the supply channel.

10. The inhalation device according to claim 1, wherein the sensor is configured to detects a situation indicative of that an orientation of the inhalation device is substantially horizontal the orientation being measured between a main axis along a longitudinal extension of the inhalation device and a normal to ground.

11. The inhalation device according to claim 1, wherein the sensor is configured to detect a situation indicative of that an orientation of the inhalation device is in a range of more than 60 degrees to horizontal, the orientation being measured between a main axis along a longitudinal extension of the inhalation device and a normal to ground.
